(19) United States
(12) Patent Application Publication
(10) Pub. No.: US 2008/0095300 A1
(43) Pub. Date: Apr. 24, 2008

(54) System and method for iterative reconstruction using parallel processing

(75) Inventors: Stephen Eric Zingelewicz, Scotia, NY (US); Austars Raymond Schnore, Scotia, NY (US); Walter Vincent Dixon, Delanson, NY (US); Samit Kumar Basu, Niskayuna, NY (US); Bruno De Man, Clifton Park, NY (US); William D. Smith, Schenectady, NY (US)

Correspondence Address: GENERAL ELECTRIC COMPANY (PCPI);C/O FLETCHER YODER, P. O. BOX 692289, HOUSTON, TX, 77269-2289, US

(73) Assignee: General Electric Company

(21) Appl. No.: 11/543,377

(22) Filed: Oct. 5, 2006

(51) Int. Cl. H05G 1/60; A61B 6/00; G01N 23/00; G21K 1/12

(52) U.S. Cl. 378/4

(57) ABSTRACT

A method and system are provided for processing an acquired image signal in parallel to generate a reconstructed image signal. In one embodiment, a processing component is provided comprising one or more field-programmable gate arrays configured as co-processors. Other aspects of the present technique provide a pipelined processor configured to forward- and back-project image data using the same data path and arithmetic units.

### BACKGROUND

The invention relates generally to medical imaging and, more specifically, to the iterative reconstruction of medical images.

Non-invasive imaging broadly encompasses techniques for generating images of the internal structures or regions of a person or object that are otherwise inaccessible for visual inspection. One of the best known uses of non-invasive imaging is in the medical arts where these techniques are used to generate images of organs and/or bones inside a patient which would otherwise not be visible. Example of such non-invasive imaging modalities include X-ray based techniques, such as computed tomography (CT).

CT scanners operate by projecting fan shaped or cone shaped X-rays from an X-ray source. The X-ray source emits X-rays at numerous angles relative to an object being imaged, such as a patient, which attenuates the X-rays as they pass through. The attenuated X-rays are detected by a set of detector elements, which produce signals representing the attenuation of the incident X-rays. The signals are processed and reconstructed to form images which may be evaluated themselves or which may be associated to form a volume rendering or other representation of the imaged region. In a medical context, pathologies or other structures of interest may then be located or identified from the reconstructed images or rendered volume.

CT reconstruction is usually performed using direct reconstruction techniques based on mathematical ideals that are not typically observed in practice. One side effect of the failure of the mathematical ideals to correspond to actual practice is that noise and resolution performance for a given X-ray dose is typically not optimized using direct reconstruction techniques.

Iterative reconstruction techniques overcome these problems by employing various mathematical models, such as noise and system models, to account for deviations from the mathematical ideals. Iterative reconstruction techniques repeatedly apply respective forward and backward projection models to generate an image that best fits the measured data according to an appropriate objective function. In this manner, iterative reconstruction algorithms may provide improved image quality and/or reduced X-ray dosage. In addition, iterative reconstruction algorithms may provide other benefits, such as reduction of metal artifacts in reconstructed images.

However, iterative reconstruction algorithms require significantly more computation than conventional, i.e., direct, reconstruction methods and have thus far been impractical for mainstream CT applications. In particular, iterative reconstruction algorithms undergo many iterations to generate each image, i.e., to converge. Further, each iteration employs two or more computationally intensive projection and back-projection operations. As a result, iterative reconstruction algorithms may require an order of magnitude or more computational effort than a direct reconstruction technique. Consequently, iterative reconstruction approaches are typically much slower than comparable direct reconstruction approaches. As a result, real time or near-real time implementations of iterative reconstruction techniques have not been practical.

### BRIEF DESCRIPTION

A tomographic imaging system is provided in accordance with one aspect of the present technique. The tomographic imaging system includes a processing component comprising one or more field programmable gate arrays configured as co-processors to a microprocessor.

A method for reconstructing a tomographic image is provided in accordance with the present technique. The method includes processing an acquired image signal using one or more field programmable gate arrays configured as co-processors to a microprocessor to generate a reconstructed image signal.

A tomographic imaging system is provided in accordance with one aspect of the present technique. The tomographic imaging system includes means for iteratively reconstructing a tomographic image using parallel processing.

A tomographic imaging system is provided in accordance with one aspect of the present technique. The tomographic imaging system includes a processing unit comprising one or more co-processing components configured to operate on an acquired image signal in parallel to generate a reconstructed image signal.

A method for reconstructing a tomographic image is provided in accordance with the present technique. The method includes processing an acquired image signal using one or more co-processor components operating in parallel to generate a reconstructed image signal.

A pipelined processor is provided in accordance with one aspect of the present technique. The pipelined processor is configured to forward- and back-project image data using the same data path and arithmetic units.

A method for processing image data is provided in accordance with the present technique. The method includes performing projection and back-projection operations on a pipelined processor. The projection and back-projection operations employ the same data path and arithmetic units.

A view subset pipeline is provided in accordance with one aspect of the present technique. The view subset pipeline includes a plurality of pipelined processors. The view subset pipeline also includes a plurality of respective memories in communication with the plurality of pipelined processors.

A method for processing image data is provided in accordance with the present technique. The method includes providing image data to a view subset pipeline. The method also includes processing the image data using a plurality of pipelined processor of the view subset pipeline.

### DRAWINGS

These and other features, aspects, and advantages of the present invention will become better understood when the following detailed description is read with reference to the accompanying drawings in which like characters represent like parts throughout the drawings, wherein:

FIG. 1 is a diagrammatical view of an exemplary imaging system in the form of a CT imaging system for use in producing images in accordance with aspects of the present technique;

FIG. 2 is a diagrammatical view of an exemplary processing component for use in the imaging system of FIG. 1 in accordance with aspects of the present technique;

FIG. 3 is a flowchart depicting exemplary logic for implementing a portion of an MLTR iterative reconstruction algorithm, in accordance with the present technique;

FIG. 4 is a flowchart depicting exemplary logic for implementing a further portion of an MLTR iterative reconstruction algorithm, in accordance with the present technique; and

FIG. 5 is a diagrammatical view of an exemplary view subset pipeline in accordance with aspects of the present technique.

### DETAILED DESCRIPTION

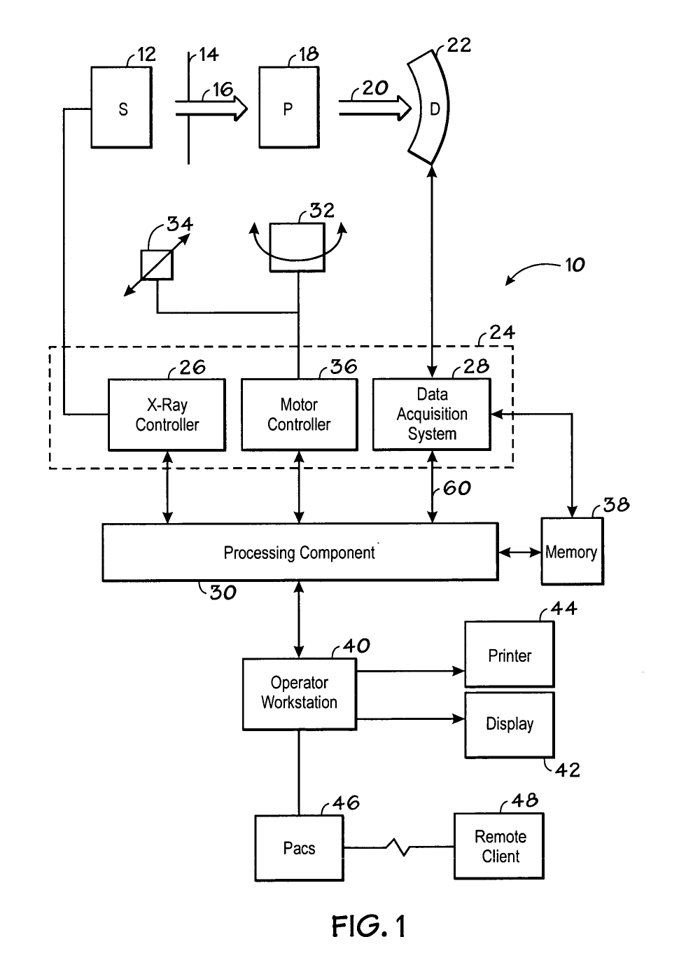

FIG. 1 illustrates diagrammatically an imaging system 10 for acquiring and processing projection data to produce reconstructed images. In the illustrated embodiment, system 10 is a computed tomography (CT) system designed both to acquire original image data, and to process the image data for display and analysis in accordance with the present technique. The system 10 is configured to employ iterative reconstruction techniques in the processing of acquired or saved projection data to reconstruct medically useful images in accordance with the present technique. In the embodiment illustrated in FIG. 1, imaging system 10 includes a source of X-ray radiation 12 positioned adjacent to a collimator 14. In one exemplary embodiment the X-ray source 12 is an X-ray tube. In other embodiments the X-ray source 12 may be a distributed X-ray source, such as a solid-state or thermionic X-ray source, or may be other sources of X-ray radiation suitable for the acquisition of medical images.

The collimator 14 permits X-rays 16 to pass into a region in which an object, such as a subject of interest 18, is positioned. A portion of the X-ray radiation 20 passes through or around the subject and impacts a detector array, represented generally at reference numeral 22. Detector elements of the array produce electrical signals that represent the intensity of the incident X-rays 20. These signals are acquired and processed to reconstruct images of the features within the subject 18.

Source 12 is controlled by a system controller 24, which furnishes both power, and control signals for CT examination sequences. In the depicted embodiment, the system controller 24 controls the source 12 via an X-ray controller 26 which may be a component of the system controller 24. In such an embodiment, the X-ray controller 26 may be configured to provide power and timing signals to the X-ray source 12.

Moreover, the detector 22 is coupled to the system controller 24, which commands acquisition of the signals generated in the detector 22. In the depicted embodiment, the system controller 24 acquires the signals generated by the detector using a data acquisition system 28. In this exemplary embodiment, the detector 22 is coupled to the system controller 24, and more particularly to the data acquisition system 28. The data acquisition system 28 receives data collected by readout electronics of the detector 22. The data acquisition system 28 typically receives sampled analog signals from the detector 22 and converts the data to digital signals for subsequent processing by a processor 30 discussed below. The system controller 24 may also execute various signal processing and filtration functions with regard to the acquired image signals, such as for initial adjustment of dynamic ranges, interleaving of digital image data, and so forth.

In the embodiment illustrated in FIG. 1, system controller 24 is coupled to a rotational subsystem 32 and a linear positioning subsystem 34. The rotational subsystem 32 enables the X-ray source 12, collimator 14 and the detector 22 to be rotated one or multiple turns around the subject 18. It should be noted that the rotational subsystem 32 might include a gantry. Thus, the system controller 24 may be utilized to operate the gantry. The linear positioning subsystem 34 enables the subject 18, or more specifically a table, to be displaced within an opening in the CT system 10. Thus, the table may be linearly moved within the gantry to generate images of particular areas of the subject 18. In the depicted embodiment, the system controller 24 controls the movement of the rotational subsystem 32 and/or the linear positioning subsystem 34 via a motor controller 36.

In general, system controller 24 commands operation of the imaging system 10 (such as via the operation of the source 12, detector 22, and positioning systems described above) to execute examination protocols and to process acquired data. For example, the system controller 24, via the systems and controllers noted above, may rotate a gantry supporting the source 12 and detector 22 about a subject of interest so that a plurality of radiographic views may be collected for processing. In the present context, system controller 24 also includes signal processing circuitry, typically based upon a general purpose or application-specific digital computer, associated memory circuitry for storing programs and routines executed by the computer (such as routines for executing image processing and reconstruction techniques described herein), as well as configuration parameters and image data, interface circuits, and so forth.

In the depicted embodiment, the image signals acquired and processed by the system controller 24 are provided to a processing component 30 for reconstruction of images. The processing component 30 may be one or more conventional microprocessors. In one embodiment, the processing component 30 has a scalable architecture, such as may be suitable for an exemplary field programmable gate array implementation, discussed herein. The data collected by the data acquisition system 28 may be transmitted to the processing component 30 directly or after storage in a memory 38. It should be understood that any type of memory to store a large amount of data might be utilized by such an exemplary system 10. Moreover, the memory 38 may be located at the acquisition system site or may include remote components for storing data, processing parameters, and routines for iterative image reconstruction described below.

The processing component 30 is configured to receive commands and scanning parameters from an operator via an operator workstation 40 typically equipped with a keyboard and other input devices. An operator may control the system 10 via the input devices. Thus, the operator may observe the reconstructed images and/or otherwise operate the system 10 via the operator workstation 40. For example, a display 42 coupled to the operator workstation 40 may be utilized to observe the reconstructed images and to control imaging. Additionally, the images may also be printed by a printer 44 which may be coupled to the operator workstation 40.

Further, the processing component 30 and operator workstation 40 may be coupled to other output devices, which may include standard or special purpose computer monitors and associated processing circuitry. One or more operator workstations 40 may be further linked in the system for outputting system parameters, requesting examinations, viewing images, and so forth. In general, displays, printers, workstations, and similar devices supplied within the system may be local to the data acquisition components, or may be remote from these components, such as elsewhere within an institution or hospital, or in an entirely different location, linked to the image acquisition system via one or more configurable networks, such as the Internet, virtual private networks, and so forth.

It should be further noted that the operator workstation 40 may also be coupled to a picture archiving and communications system (PACS) 46. It should be noted that PACS 46 might be coupled to a remote client 48, radiology department information system (RIS), hospital information system (HIS) or to an internal or external network, so that others at different locations may gain access to the image, the image data, and optionally the variance data.

While the preceding discussion has treated the various exemplary components of the imaging system 10 separately, one of ordinary skill in the art will appreciate that these various components may be provided within a common platform or in interconnected platforms. For example, the processing component 30, memory 38, and operator workstation 40 may be provided collectively as a general or special purpose computer or workstation configured to operate in accordance with the present technique. Likewise, the system controller 24 may be provided as part of such a computer or workstation.

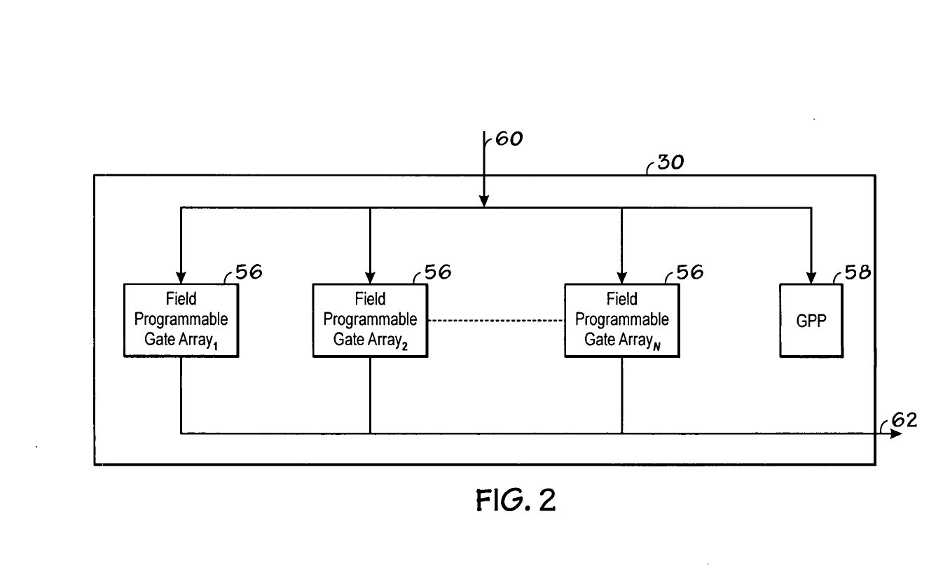

In one embodiment of the present technique, the processing component 30 consists of one or more general purpose processors (GPP), such as central processing units or microprocessors found in a general purpose computer or workstation, which are capable of implementing an iterative reconstruction algorithm. Such GPPs may be high-speed and readily commercially available. In another embodiment, depicted by FIG. 2, the processing component 30 may include one or more custom processors configured as components of field programmable gate arrays (FPGA) 56 which act as co-processors to a GPP 58. In such an embodiment, an FPGA board 56 may contain several custom reconstruction units that operate on the acquired image signals 60 in parallel to generate a reconstructed image signal 62 using iterative techniques. In such an implementation, the iterative reconstruction algorithm may be implemented by the FPGAs such that multiple stages of the iterative reconstruction or independent operations and/or updates are performed in parallel.

For example, in one embodiment, the parallel architecture implemented using FPGA boards 56 may contain up to twenty-two reconstruction units in the form of fully pipelined processors. In one such an embodiment, each reconstruction unit can process up to one pixel/detector boundary per clock cycle or otherwise provides near complete utilization of the arithmetic units provided by the processors on the FPGA boards 56. Such an embodiment is linearly scalable across multiple FPGA boards 56 and may be included as a hardware-based reconstruction accelerator in a general purpose computer or workstation. Indeed, such an embodiment may perform two iterative image reconstructions per second or more.

In one implementation of such an embodiment, the parallel processing provided by the FPGA-mounted reconstruction units is used to execute a suitable projection algorithm (such as the distance driven projector algorithm discussed below) in stages. These stages may be designed and configured for such a hardware implementation to provide efficient computation of the various operations of the projection algorithm. For example, the various stages of the projection algorithm may be executed concurrently once the data pipeline is filled with data. In this manner, the overall speed and efficiency of the calculation may be improved by processing several pieces of data at a time. Furthermore, the projection algorithm implemented by the hardware may be used for both forward projection (generating a sinogram from an image) and backward projection (generating an image from a sinogram). In such a hardware implementation, the hardware resources may be fully or substantially utilized by using the same datapath and arithmetic units for both forward and backward projection operations. An exemplary implementation of such an embodiment is discussed in greater detail below.

In selecting a suitable projection algorithm for use in such a hardware implementation, one of ordinary skill in the art will appreciate that back projection and re-projection operations are important components of an iterative reconstruction algorithm. A suitable projection algorithm should, therefore, execute efficiently and minimize high frequency artifacts to allow rapid iteration of the projection operations while providing suitable image quality.

Examples of projection methods include methods that are pixel-driven or ray-driven. Fundamentally both the pixel-driven and ray-driven algorithms re-sample the sinogram or image values as a function of detector channels or pixels (respectively). For example, pixel-driven back-projection projects a line from the focal spot through the center of the image pixel of interest onto the detector array using the imaging geometry. Once a location of intersection on the detector is determined, a value is obtained from the detector (such as by linear interpolation) and the result is accumulated in the image pixel. In such a back-projection approach, a sinogram row is the source signal and an image row is the destination signal. For each image row, the pixel centers are mapped on to the detector. Pixel-driven projection is the transpose operation of the back-projection operation described above. Pixel-driven techniques are so named because the index of the main processing loop is the image pixel index.

Conversely, ray-driven projection generally consists of approximating each ray-integral by weighting and summing all image pixels that lie close to the ideal projection line. The ideal projection line may be approximated by projecting a line from the focal spot through the image to the center of the detector element of interest using the imaging geometry. A location of intersection is calculated for each image row (or column), a value is obtained from the image row, such as by linear interpolation, and the result is accumulated in the detector element. In such a projection approach, an image row is the source signal and a sinogram row is the destination signal. For each image row, the detector element centers are mapped on to the image row. Ray-driven back-projection is the transpose operation of the projection operation described above. In ray-driven techniques the index of the main processing loop is the projection line index. While ray- and pixel-driven techniques have certain advantages, other projection methods may be as or more suited for implementation in a hardware accelerator.

For example, an alternative projection algorithm suitable for use in a hardware implementation as described herein is a distance-driven projection algorithm. The distance-driven projection technique can be summarized as the mapping of pixel and detector coordinates onto a common line or axis followed by a kernel operation. Distance-driven techniques are based upon the recognition that each view (i.e., source position) defines a bijection between the position on the detector and the position within an image row or column. Therefore, every point within an image row or column is mapped uniquely onto a point on the detector and vice versa. A length of overlap between each image pixel and detector element may, therefore, be defined. This overlap may be calculated by mapping all pixel boundaries in an image row or column of interest onto the detector or by mapping all detector element boundaries of interest onto the centerline of the image or column row of interest. In one embodiment, this is accomplished by mapping both image pixel and detector element boundaries onto a common line or axis by connecting all pixel boundaries and all detector element boundaries with the source and calculating the intercepts on the common axis. Based on these calculated intercepts, the length of overlap between each image pixel and each detector element can be calculated as seen on the common axis. A one-dimensional kernel operation may then be applied to map data from one set of boundaries to the other. The normalized length of overlap between each image pixel and detector cell may be used to define the weight used in projection and back-projection processes. The distance driven projection algorithm is well suited for iterative reconstruction and can be efficiently implemented in hardware. The distance-driven projection algorithm performs both forward-projection and back projection operations without artifacts, has low arithmetic complexity, and provides for sequential memory accesses. In addition, the distance-driven projection algorithm is symmetric with regard to the forward-projection and back-projection operations performed, allowing hardware resource sharing in a hardware implementation. As will be appreciated by those skilled in the art, this matched projector-backprojector pair is also useful to avoid image artifacts with iterative reconstruction. While the preceding discussion is explained in terms of two-dimensional projection/backprojection for simplicity, the concepts discussed are extendable to three-dimensions, as would be understood by those of ordinary skill in the art. In such three-dimensional contexts, the concepts of a pixel should be understood as also encompassing corresponding three-dimensional constructs, such as voxels. Likewise, in the three-dimensional context the length of overlap corresponds to area of overlap and so forth.

As will be appreciated by those of ordinary skill in the art, selection of a suitable projection algorithm is only one aspect of implementing an iterative reconstruction algorithm in hardware. Another aspect of such an implementation is the selection of a suitable iterative reconstruction algorithm. Two such iterative reconstruction algorithms are the iterative coordinate descent (ICD) algorithm and the maximum likelihood for transmission tomography (MLTR) algorithm. The MLTR algorithm is included in this discussion as a representative of a broad class of simultaneous update approaches, including other ordered subset algorithms as well as for exemplary conjugate gradient-based algorithms. Both the ICD and MLTR algorithms require efficient forward-projection and back-projection algorithms and so may benefit from selection of a suitable projection algorithm, such as the distance-driven projection algorithm discussed above.

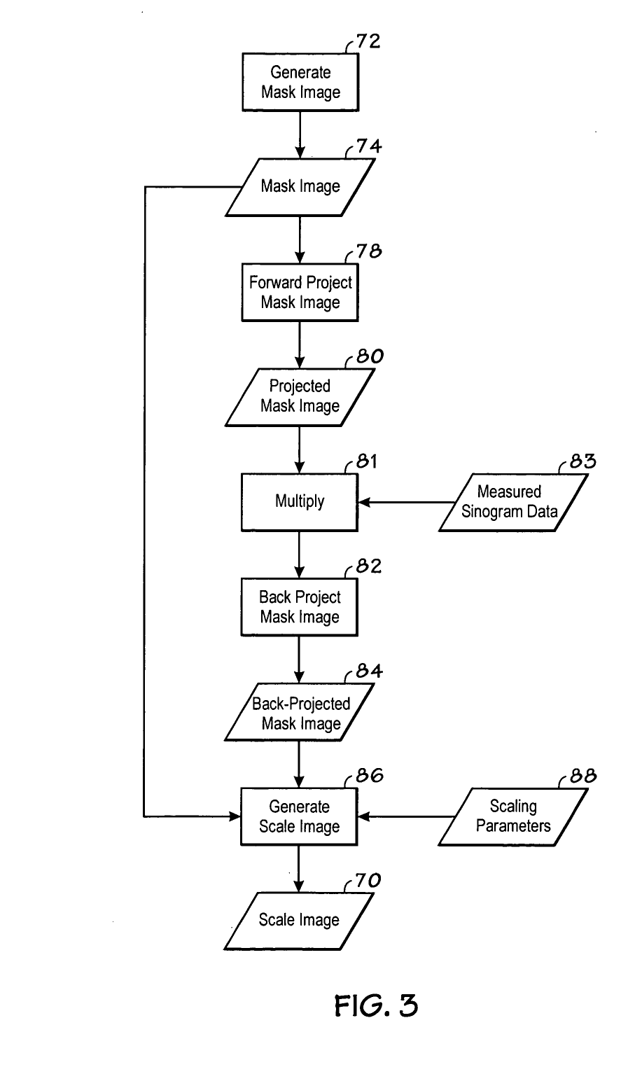

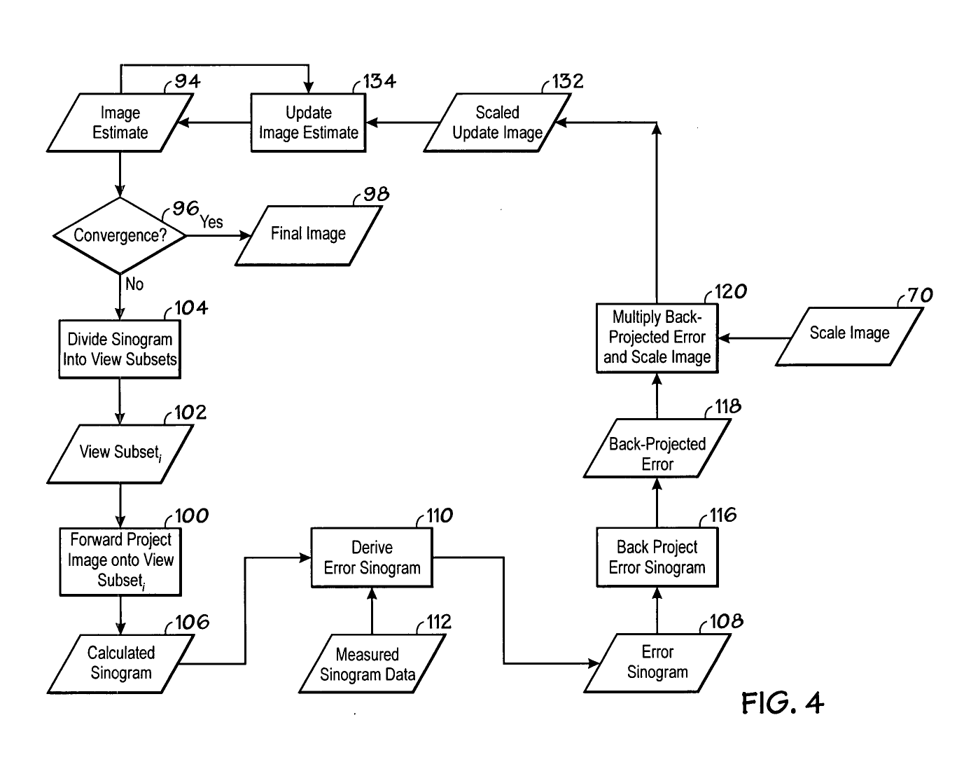

Exemplary logic for iteratively implementing the MLTR algorithm is provided in FIG. 3, depicting the generation of scale image 70 for use in iterative processing, and FIG. 4, depicting iterative processing using the scale image 70 of FIG. 3. With regard to FIG. 3, the algorithm begins with the generation (block 72) of a mask image 74. The mask image 74 defines the subset of the image pixels that will actually participate in the reconstruction. In one implementation, the mask image 74 is a binary image where a value of one is assigned to pixels that will be active and a value of zero is assigned to pixels that will not be active in the reconstruction process. For example, in typical object scans a large fraction of the pixels in the image are outside the object, and thus may be excluded from the mask image 74 by assigning a value of zero to these pixels. For instance, in medical scanning, pixels that are outside the actual patient may be excluded from the image mask 74 by assigning a value of zero to these external pixels. Similarly, for industrial scanning, the mask image 74 may exclude pixels that are outside the object being scanned. Further, objects with a high aspect ratio (such as a patient's shoulders or a turbine blade) will have a larger fraction of these zero pixels. The mask image 74 may be generated at block 72 from any number of sources, including a lower resolution direct reconstruction of the measured sinogram data, from the projection boundaries themselves (which can be back-projected and intersected to form a convex mask), or from related datasets (such as a neighboring slice). The mask image 74 may be larger than the actual object and its supporting structure, and may be replaced with a suitable analytic shape, such as an ellipse.

In the depicted implementation, the mask image 74 is projected (block 78) using a model of the system that incorporates physical, geometric, or physics-based aspects of the imaging system. The result of this projection step is a projected mask image 80 which may be subsequently processed. Projection may be accomplished using distance-driven forward projection (as discussed herein), ray-driven forward projection, splatting, or other methods. In an implementation where the mask image 74 is a binary image having pixel values of one and zero, the projection at block 78 may be particularly simplified. For example, if the boundary is known to be an ellipse, the projection can be calculated directly from a mathematical formula instead of actually projecting pixels.

In one embodiment, the projected mask 80 may be multiplied by a measured or calculated sinogram. For example, in the depicted embodiment, the projected mask image 80 is multiplied (block 81) by the measured sinogram data 83. The projected mask image 80 or its product is then back-projected (block 82), such as via distance-driven back-projection, to generate a back-projected mask image 84. The back-projected mask image 84 is used to generate (block 86) the scale image 70. For example, in one embodiment, the back-projected mask image 84 is inverted (that is, each pixel is assigned a value of 1/x, where x is the current pixel value) and multiplied pixelwise by the mask image 74. The product of this combinatorial step may then be scaled using one or more scaling parameters 88. For example, in one embodiment, the product of the mask image 74 and the inverted back-projected mask image 84 is multiplied by the number of views in the dataset and a relaxation factor, alpha, that is less than two. The result is stored as the scale image 70. As will be appreciated by one of ordinary skill in the art, the steps depicted in FIG. 3 directed to the generation of the scale image 70 encompass the setup or preliminary stages of the reconstruction algorithm.

Turning now to FIG. 4, steps constituting the iterative part of an exemplary reconstruction are depicted. In the depicted embodiment, the process is initialized with an estimate image 94, which may be an image from a direct reconstruction of the measured sinogram data or an image of all zeros, for example. In instances where the image estimate 94 has converged, as determined at decision block 96, the converged estimate may be output as a final image 98. In one embodiment, the algorithm is considered converged when the desired spatial resolution for the final image 98 is reached. However, as will be appreciated by those of ordinary skill in the art, other stopping criteria may also be employed.

In one embodiment, view subsets 102 are generated (block 104) when the image estimate 94 is not converged and the MLTR algorithm leverages the view subsets 102 to accelerate image reconstruction. In one embodiment the view subsets 102 are selected based on orthogonal set of views such that each subset 102 consists of a uniform sample from the complete view set. For example, in one embodiment, there may be 984 total views and an average subset size of approximately 10, with a maximum subset size of 11 and a minimum subset size of 9. In such an embodiment, there may be approximately 95 view subsets 102. In general, the view subsets 102 are taken sequentially to cover all of the views in the sinogram and are chosen so that, for example, each subset 102 is roughly the same size, and the views in each subset are maximally distant from each other. Other techniques for generating view subsets 102 may also be employed, however. As discussed below, in embodiments employing view subsets, a subsequent scaling process may also be employed.

In the depicted embodiment, a current view subset 102 of the current image estimate 94 is projected (block 100). In one embodiment, the projection is done using a distance-driven algorithm or any other suitable forward projector. The resulting sinogram is called the calculated sinogram 106. An error sinogram 108 is derived (block 110) from the calculated sinogram 106. The error sinogram may be generated by various techniques. For example, in an exemplary implementation, views of the measured sinogram data 112 that correspond to the current view subset 102 are subtracted from the calculated sinogram 106 to generate the error sinogram 108. In such an implementation, the measured sinogram data 112 can be log corrected and in line-integral attenuation form. In other embodiments, the derivation of the error sinogram 108 may be based upon a suitable statistical model, such as a Poisson or least squares model.

In the depicted embodiment, the error sinogram 108 is back-projected (block 116, such as via distance-driven back-projection or any other suitable back-projector, to form the back-projected error 118. The back-projected error image 118 is multiplied (block 120) pixelwise by the scale image 70. In one embodiment, computation may be saved or reduced by not back-projecting onto pixels that are zeroed out by this operation. In one embodiment, the resulting update image is scaled by 1/(number of views in the current view subset) to generate a scaled update image 132. In an alternative embodiment, the scale image 70 is prescaled by 1/Nmax, where Nmax is the size of the largest view subset. In such an embodiment, no further scaling is necessary as the scaled update image 132 is the product of the multiplication step of block 120.

As will be appreciated by those of ordinary skill in the art, a scaling process as described above may be appropriate in embodiments employing view subsets 102. In particular, when employed, view subsets 102 reduce the amount of sinogram data required in a projection process. In addition, the use of view subsets 102 may reduce computation time because only one subset of the views is evaluated at a time in the projection and back projection cycles. One implication on the baseline MLTR algorithm is that the resulting calculated sinogram 106 and error sinogram 108 are proportional to the size of the current view subset 102, not to the total number of views. A second implication, therefore, is that the resulting update image is subsequently scaled when necessary to compensate for the reduced number of views per subset, thereby generating the scaled update image 132. Thus each iteration (consisting of several view subset iterations) still involves processing every view of the respective subsets, but the total number of iterations required for an image to converge is dramatically reduced. As will be appreciated by those of ordinary skill in the art, increasing the view subset size reduces the number of subsets, but typically increases the number of iterations required.

Returning now to FIG. 4, the scaled update image 132 is used to update (block 134) the image estimate 94. For example, in one embodiment, the scaled update image 132 is subtracted from a current image estimate to form an updated image estimate. The process depicted by FIG. 4 may be repeated at different view subsets 102 until convergence occurs as determined at decision block 96 and a final image 98 is obtained.

As will be appreciated by those of ordinary skill in the art, the MLTR calculations (projection and back-projection, image scaling, and the error calculations) may be fully pipelined to yield a result on every processor clock cycle. As will be appreciated by those of ordinary skill in the art, pipelining is the process of executing multiple stages of an operation at the same time over multiple pieces of data. With fully pipelined calculations, the baseline MLTR would require approximately 1.5×10⁹ clock cycles per iteration. At 46 iterations to converge and using a 100 MHz FPGA, this would amount to roughly 689 seconds for a single image to converge. Implementations employing algorithm improvement and/or hardware parallelization may reduce this convergence time, however.

For example, improved algorithm performance may be obtained by calculating the error sinogram 108 using a least squares or weighted least squares calculation, i.e., an LSTR implementation. Similarly, over-relaxation may be employed in the iterative reconstruction algorithm and/or the field-of-view mask employed for reconstructed images may be tightened or otherwise reduced. Speed of convergence of the iterative reconstruction algorithm may be further increased by reducing the size of the image being reconstructed, such as by sizing the image to match the field of view mask. Incorporation of these types of modifications into the MLTR algorithm may allow convergence of a final image 98 within four iterations or less with a 50% noise reduction. Thus—based on 1.5×10⁹ clock cycles per iteration—a hardware accelerator running at 100 MHz would require approximately 60 seconds for a typical image to converge. Note that these timing examples assume that no regularizer (or equivalently, no prior) is used and that the algorithm is converged when the desired spatial resolution is reached.

With regard to hardware acceleration, the MLTR iterative reconstruction algorithm is well suited for hardware implementation using a suitable projection algorithm, such as the distance-driven projection algorithm discussed herein. For example, in such an implementation, the same distance-driven projector hardware may be used in both the forward-projection and back-projection operations. In terms of hardware acceleration, parallelism in the distance-driven projector units can be exploited. In such an embodiment, during forward and re-projection the same image data may be used by each distance-driven projector unit to form views in a subset. Hence the streaming of the image data provides an opportunity for parallelization of the MLTR and distance-driven projection algorithms in hardware.

For example, in one embodiment, the distance driven projectors may be provided as a pipeline configured to iterate over each view in a respective subset and which can be unrolled. In such an embodiment, the speed increase is proportional to the number of views per subset. In particular, multiple distance-driven projector units can be employed to handle all views concurrently within the subset. This is equivalent to unrolling the loop for iterating on views in a subset. Furthermore, the image scaling and update calculations can be folded into the same back-projection image stream. In such an embodiment, the set of distance-driven projector units may be implemented as a view subset pipeline. In one embodiment of such an implementation, sufficient calculation density on the FPGA hardware may be achieved by porting the reconstruction algorithm from single precision floating point to 32 bit fixed point calculations. Such a conversion has been observed not to significantly degrade image quality or to affect the number of iterations required for convergence.

In an embodiment that streams pixel data to the distance-driven projector units in this manner, images are provided at the same orientation to all distance-driven projector units. When working with a single distance-driven projector unit, the image may be oriented to maximize the view angle with respect to the image row or column. Thus normal image orientations are used for view angles from 315° to 45° and from 135° to 225° and rotated images are used for view angles between 45° and 135° and between 225° and 315°. In a parallel implementation as discussed above, however, the view subset definitions are modified to use “one-sided subsets” that consist either of views that all have normal orientation or views that are all rotated by 90°. The use of these one-sided subsets may facilitate a pipelined implementation by ensuring that all views are computed on an image in a single orientation, however embodiments which use such a modified view subset definition may take an additional iteration (i.e., 5 iterations) for an image to converge.

The algorithm and hardware modifications described above may provide a speed-up proportional to the average view subset size and may bring the time to converge a single image down to roughly 7.5 seconds (again assuming a 100 MHz system clock rate in the accelerator hardware). In addition, the system is scalable so that more distance-driven projector units may be added as view subset sizes are increased. However, as will be appreciated by those of ordinary skill in the art, larger subset sizes may hurt convergence, therefore it may be desirable to limit how many distance-driven projector units can be configured in a view subset pipeline unit.

In an embodiment where the distance-driven projector units operate on image rows independently of each other, processing speed may be increased by processing sub-images on different view subset pipeline units operating in parallel. In such an embodiment, the resulting view information from each view subset pipeline during re-projection is combined by summing across the corresponding detector channels to form a single view. Therefore, in implementations where a single FPGA device holds up to two view subset pipeline units, synchronization across multiple FPGA devices is provided.

Alternatively, a round-robin scheduling scheme with each view subset pipeline unit computing a single image from a sinogram may be implemented. Such a round-robin scheduling scheme is scalable and the frame rate may be scaled linearly by adding additional FPGA devices to the system. Given equivalent view subset pipeline resources the image throughput (frames per second) should be essentially the same in both embodiments.

The exemplary hardware implementations discussed herein may achieve convergence in 7.5 seconds per image on a single view subset pipeline in a system that runs at 100 MHz. Furthermore, it is believed each FPGA device can fit two view subset pipeline units and that newer, faster FPGA devices running at a 200 MHz system clock will allow a sustained frame rate of just over 2 frames per second on a card with four such FPGA devices. As will be appreciated by those of ordinary skill in the art, further increases in scaling and/or processor speed may be leveraged to further increase the sustained frame rate.

While MLTR reconstruction algorithms may be implemented in hardware, as described above, other reconstruction algorithms may also be implemented in accordance with the present techniques. For example, the iterative coordinate descent (ICD) family of algorithms may also be implemented in accordance with the present techniques. The baseline ICD algorithm operates by iterating over image pixels, with each pixel iterating in the inner-loop over its corresponding sinogram data track. In order to optimize convergence time and enhance potential parallelism for hardware acceleration, a number of variations on the ICD algorithm may be implemented. For example, under-relaxation, pixel subsets (also known as grouped coordinate descent), and pixel subsets with multi-resolution may be desirable implementations of the ICD algorithm in a hardware accelerated, parallel architecture as described herein. These algorithm enhancements may also improve memory access characteristics of the baseline ICD algorithm.

For example, in one embodiment relaxation factors are employed in the ICD process. In this embodiment, the use of smaller relaxation factors accelerates the initial image convergence. In another embodiment, pixel subsets are employed by the ICD algorithm. As will be appreciated by those of ordinary skill in the art, such pixel subsets are analogous to view subsets in MLTR and allow multiple pixels to be processed concurrently. In this embodiment, pixel subsets may be selected to minimize interactions of the sinogram tracks. In other embodiments, the pixel subset approach may be combined with multi-resolution techniques. Such combination embodiments reduce image artifacts and may achieve acceptable image convergence in 5 iterations or less. In embodiments employing these modifications and employing a distance-driven projection technique, a sparse distance-driven projector and a sparse distance-driven back-projector may be provided in addition to a conventional distance-driven projector. In some embodiments, hardware resources between the sparse projector and sparse back projector may be shared in hardware. Furthermore, the conventional distance-driven projector may be synthesized as a combination of sparse distance-driven projectors.

The concepts regarding hardware and algorithm implementation noted above were implemented in an exemplary system. With regard to the reconstruction algorithm, a MLTR/LSTR reconstruction algorithm was implemented in the exemplary system in accordance with the transformations and optimizations discussed above with regard to an FPGA implementation. The hardware implementation of the MLTR/LSTR algorithm incorporated a fixed-point quantization scheme. In addition, specific elements in the functional model, such as the distance-driven projector units and the view subset pipeline, were developed in Verilog® using conventional hardware development methodology. High-level language based synthesis tools were used to create the top-level data and control flow design for the algorithm, manage host communications over PCI, and coordinate external memory accesses.

The exemplary implementation of the MLTR/LSTR algorithm in hardware leveraged commercial-off-the-shelf development hardware, IP cores, and communications services. In this implementation, a conventional Xilinx® development tool flow was employed. This tool was augmented with high-level language based synthesis tools that allowed us to compile high-level functional models down to register-transfer level implementations of the algorithm. Several components of the MLTR/LSTR algorithm were implemented in Verilog® using traditional hardware development methods and then folded into the synthesis tools as IP cores.

The FPGA development hardware used in the exemplary implementation was from Nallatech®. For example, BenNUEY® cards from Nallatech® were employed which are PCI development boards that allows FPGA devices to be configured over the PCI bus from the host Linux or Windows system. The BenNUEY® PCI card implements a 64-bit 33 MHz PCI interface. The BenNUEY® card has three sites for daughter DIME-II modules and Nallatech offers a variety of such DIME-II daughter modules allowing developers to configure the development hardware to match the characteristics of the intended applications. We used three BenDATA_WS modules for use in the MLTR/LSTR algorithm implementation. The BenNUEY® card has a user configurable FPGA device with two small banks of ZBT SRAM memory. We used this FPGA device to route communications between the host computer and the MLTR/LSTR accelerators deployed in the FPGA devices on the three daughter cards. The card architecture provides a variety of communications paths between the user FPGA on the card and among the DIME-II modules. With the round-robin based MLTR/LSTR implementation, communication from the carrier card was limited to communication between the user FPGA and each of the BenDATA_WS modules.

Each BenDATA_WS module consisted of a single FPGA device (Xilinx® Virtex-II® 6M with 1152 pin packaging, speed grade 4) connected to 6 independent banks of 64 bit wide ZBT SRAM memory with 4 Mbytes in each of the memory banks (24 Mbytes total). As implemented, the MLTR/LSTR algorithm used 32 bit data values (or less). Therefore, if 32 bit wide ZBT SRAM memory banks were used and the communication paths between FPGA devices on the carrier card were reduced, a lower capacity FPGA device family (e.g., Xilinx Spartan-III®) could be used instead in a custom purpose accelerator board.

The exemplary implementation also employed ZBT SRAM memory controllers from Nallatech® as well as a variety of communication hardware primitives to implement host I/O and control. An initial floating-point version of the MLTR/LSTR algorithm also used a Nallatech® library of IEEE compatible floating operators. In later implementations, however, but this library was not needed with the fixed-point version of the MLTR/LSTR algorithm.

A packet-based routing network that suitable for host communications (control and data to/from host) was implemented in the exemplary system using the DIMETalk® tool provided by Nallatech®. The DIMETalk® tool allowed routing networks for communicating data between host and the FPGA devices on the carrier card (including the daughter cards) to be built. The DIMETalk® tool also provides API libraries for integrating the FPGA devices into host resident application software. As will be appreciated by those of ordinary skill in the art, the tools used to develop the exemplary implementation described herein provide an acceptable implementation. However, performance of an implemented system may be improved by using a custom accelerator board, such as a board having fewer pins per FPGA package, more suitable memory configurations, and so forth.

Furthermore, a custom architecture can take advantage of an algorithm's inherent parallelism and data access patterns. Loops which operate serially on a general purpose processor (GPP) can be unrolled and laid out in parallel in a custom design. For the MLTR/LSTR algorithm, the loop that processed each view in a subset was unrolled and became a set of eleven projectors. These projectors, fed off of a common pixel stream, ran in parallel to compute the data for up to eleven views in a single projection loop.

Custom architectures also benefit from being able to create an algorithm specific pipeline. As noted above, pipelining is the process of executing multiple stages of an operation at the same time over multiple pieces of data. The MLTR/LSTR hardware implementation's pipeline, while fixed, was built specifically for the MLTR/LSTR algorithm so that the processing elements have a continuous stream of data to process. Implementing the exemplary system with pipelines, in addition to processing in parallel, provided improved performance relative to execution on a GPP.

At the highest level, communication between the reconstruction hardware and the host computer was performed through remote method invocation (RMI). Hardware services were exposed to the host computer as a set of software API calls. When the computer control software executes an exposed method, this invokes a function that provides the method's parameters as a data stream that is sent to the hardware. Three classes of functions were exposed to the computer from the hardware: initialization functions, control functions, and status functions. The initialization functions configured the memory banks in the hardware with data such as the measured sinogram data, boundary geometry (i.e., the locations of the pixel and/or detector boundaries), and subset definitions. The control functions were used at runtime to control the operation of the view subset calculation pipeline and the iteration controller. The status functions allowed the PC to read back the results stored in the hardware memory banks and monitor the running hardware pipelines.

In the exemplary system, the RMI layer was built upon Nallatech's® DIMETalk® network. In particular, a DIMETalk® network was configured to provide a bidirectional 32-bit wide stream between host and hardware. The computer client and the hardware server modules were generated using a class interface supplied with the tool.

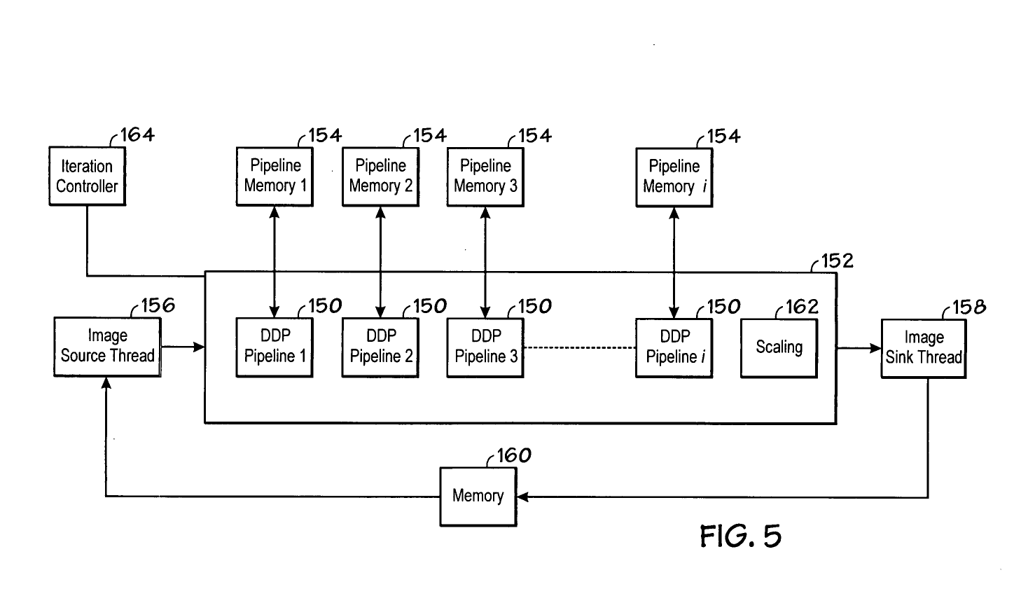

The projection/back-projection component of the exemplary system was implemented based upon a distance-driven projection algorithm, as discussed herein. The distance-driven projector module consisted of two pieces, a calculation unit, which performed forward and back projection calculations, and a wrapper, which provided control, status, and data links for the calculation unit. Referring now to FIG. 5, in the exemplary system implementation, there were eleven distance-driven projector (DDP) units 150 linked together in a chain within a view subset pipeline 152.

The exemplary wrapper consisted of a field state machine (FSM) that processed network commands and a set of BRAMs 154 to hold image data. The wrapper handled the communications with higher-level modules in the system and provided data to the calculation unit. The wrapper network was arranged as a unidirectional daisy chain where each DDP 150 has an ID number. When the network state machine received a packet, it examined the destination ID to see if the message was intended for it. If not, the packet was forwarded to the next DDP 150 in the chain. When the message arrived at the intended DDP 150, it was processed. Forwarding of messages at all the intervening DDPs continued until they received an end of sequence packet.

The DDP wrapper also performed a special function for forward projections. In a forward projection, pixel data was streamed through the DDP 150 to create a new set of view data. For each image row, view data was calculated and accumulated into the local view BRAM 154. At the end of an image, the VSP 152 requested the view data out of each DDP 150 in turn. The DDP wrapper would send the view data out the NET_OUT port to the VSP 152. Each intervening DDP 150 forwarded the data along back to the VSP 152.

In the exemplary system embodiment, the calculation unit of the DDP 150 was a fully pipelined stream processor that performed the distance-driven projection calculation. As implemented, the DDP 150 could perform both forward and back projection along with an auxiliary bypass mode. The calculation unit was optimized to process one pixel/detector boundary per clock cycle and could be stopped and restarted in mid-calculation in order to handle stall conditions in the pipeline. Based on the input geometry data, a DDP 150 could run faster or slower than its neighbor. In situations where the DDP 150 had no input pixels or where the output pipe was full, the calculation unit was configured to halt and wait for the condition to clear before continuing. This dynamic rate control may be useful when the imaging geometry requires that the overlap between detector channels and image pixels varies across the field of view. For some geometries (e.g., parallel beam data or fan beam data that has been reprocessed into parallel beam data) the overlap is uniform and dynamic rate control may not be useful.

In the implemented system, the calculation unit was composed of four internal stages. These stages corresponded to different parts of the distance driven algorithm. For example, the priming loop stage was used at the beginning of an image row and functioned to find the first detector boundary which occurs just after the first pixel boundary. Once the detector boundary was found, the priming loop stage shuts off.

Once the detector and pixel boundary positions were determined, the next stage, which performed the function of acquiring weights and values, was allowed the stage to control the geometric (MDX) and view data streams. In the implemented system this stage compared the pixel and detector boundary positions and determined which should be used in the weighting calculation. This decision was passed forward to control the operation of the following pipeline stages. This stage also generated the weights and provides these weights to the compute stage along with an accumulation value. The accumulation value is either the input pixel or view value depending on the projection mode.

In the implemented system, the compute weight stage housed the calculation unit's multiplier. The computer weight stage performed the weighting operation and passed the product and accumulation value forward to the accumulation stage. In turn the accumulate stage of the operation handled accumulating previous weighted values into a temporary buffer. Based on the decision made in the stage devoted to get weight and values, this stage output a processed pixel or view based upon the projection mode.

Referring once again to FIG. 5, when a subset was ready to be processed by the exemplary system, the view subset pipeline 152 provided the parameters a DDP 150 needed to calculate the results for a given view. The view subset pipeline 152 provided the view's geometric (MDX) and measured (view) data to the DDP 150, which the wrapper stored in the local BRAMs 154. These arrays were cached because they were used for every row in a given image. The view subset pipeline 152 then sent the geometric parameters associated with the forthcoming row to be processed. These values were provided to the calculation unit through two registers. Finally, the view subset pipeline 152 issued the start command along with the mode settings for the calculation unit and started the pixel stream. The calculation unit performed in one of three modes, forward projection, back projection, or bypass. For a back projection, measured view data was read in and used to compute an output pixel. For a forward projection, a pixel value was taken in and used to compute a view value, which was stored back into the view BRAM 154. For bypass mode, the calculation unit copies the input pixel to the output pixel stream. This mode is used when there are fewer than eleven views in a subset.

Once the calculation unit completed a row, the view subset pipeline 152 was notified and it provided a new set of row parameters for the next row. The MDX array index was reset, as the MDX data does not change during processing. The view array index was also reset for forward projection operations. In addition to resetting these indices, the network state machine also reset the calculation unit and provided it with geometric parameters for the next row. Processing continued until an entire image was completed. If a DDP 150 was operating in forward projection mode, the view subset pipeline 152 issued a command causing the network state machine to dump the contents of the view BRAM 154 back to the view subset pipeline 152.

In the implemented exemplary system, the MLTR/LSTR processing flow consisted of four basic steps: forward projection, error correction, back projection, and scaling. Each projection step consists of executing the distance-driven projection algorithm (as discussed above) and applying a weighting factor. The view subset calculation pipeline was a pipelined arithmetic core that performed the intermediate calculations between forward and back projections. These calculations consisted of a cosine-weighting step after forward projection, an error calculation step, and a cosine-weighting step before back projection. The cosine weights were pre-calculated and stored in a table for efficiency. These weights were the same for both forward and back projection and therefore could be used for both computations. The error correction was a simple subtraction between the calculated sinogram data and the measured data from the input sinogram. As implemented, the weighting calculations (2 multiplies) and the error calculation (1 subtraction for the LSTR implementation) were combined into a single hardware pipeline unit that performed calculations on every clock cycle. Hence, the time complexity of this calculation corresponded to the number of detector values in a view time the number of views in the subset plus some minor overhead for control purposes (which was 2 clock cycles per view as implemented).

Referring once again to FIG. 5, as implemented, an image source thread 156 was provided which was responsible for retrieving image pixels out of memory 160 and feeding the view subset pipeline 152. The image source thread 156 also handled corner turned memory accesses for a given subset and produced a zero pixel stream for the initialization of a back projection.

During a back-projection, the view subset pipeline produced a stream of image pixels that fed the next iteration of the algorithm. The image update thread 158 handled pulling the processed pixels from the view subset pipeline 152, scaling the processed pixels via a suitable scaling unit 162, and putting the processed pixels back into the image in external memory 160. The image update thread 158 handled corner turned memory references and controlled the memory indexing in order to place the data back into memory 160 correctly. In forward projection, the image update thread 158 was not used as the pixels coming out of the view subset pipeline 152 were only used to create view data and were not modified during processing. Therefore, there was no reason to store them back into external memory 160.

As discussed above, the view subset pipeline unit 152 of the implemented system consisted of or worked in association with: the image source thread 156; the image update thread 158, and the scaling component 162; the distance driven projector units 150 (set of DDP/DDP wrapper units configured in the DDP chain hardware); and the view subset calculation pipeline (which handled the cosine weighted sinogram multiplications and the error calculation). The number of DDPs 150 in the view subset pipeline 152 was equal to the maximum number of views in a subset (which was 11 views per subset in the implementation). The same view subset pipeline unit 152 was used for both re-projection and back-projection operations.

As will be appreciated by those of ordinary skill in the art, view subset pipeline unit 152 can be replicated in order to scale the calculation throughput on an image. For example, in one replication scenario, the view subset pipeline unit 152 is replicated with each view subset pipeline unit 152 handling a different image. In an alternative replication scenario, the image is divided into sections and each view subset pipeline unit 152 processes a section of the image.

An iteration controller 164 may also be provided in exemplary implementations to control the sequence of the processing of the view subset pipeline units 152. In such embodiments, the iteration controller controls the sequencing through the outer loop iterations used for image convergence, cycling through the view subsets within an iteration, and the operating mode (re-projection or back projection) of each view subset pipeline unit 152. In a multi-FPGA implementation, one iteration controller 164 would typically be provided for each FPGA device 56 (FIG. 2). However, in such embodiments, the iteration controller 164 can be shared among view subset pipeline units 152.

While in the present discussion reference is made to a CT scanning system in which a source and detector rotate on a gantry arrangement, it should be borne in mind that the present technique is not limited to data collected on any particular type of scanner. For example, the technique may be applied to data collected via a scanner in which an X-ray source and/or a detector are effectively stationary and an object is rotated, or in which the detector is stationary but an X-ray source rotates. Further, the data could originate in a scanner in which both the X-ray source and detector are stationary, as where the X-ray source is distributed and can generate X-rays at different locations. Further, the present technique could apply to three-dimensional or cone beam acquisitions as well as to two-dimensional acquisitions. In brief, it should be borne in mind that the system of FIG. 1 are described herein as exemplary systems only. Other system configurations and operational principles may, of course, be envisaged for acquiring and processing image data and variance data and for utilizing the data as discussed below. Furthermore, data acquired from other tomographic imaging modalities, such as data acquired by magnetic resonance imaging (particularly in projection mode imaging), generalized X-ray tomography and tomosynthesis systems, nuclear imaging or positron emission tomography systems, may be utilized in the manner discussed above.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A tomographic imaging system, comprising:
a processing component comprising one or more field programmable gate arrays configured as co-processors to a microprocessor.

2. The tomographic imaging system of claim 1, wherein each field programmable gate array comprises one or more reconstruction units configured to operate in parallel.

3. The tomographic imaging system of claim 1, wherein each field programmable gate array comprises one or more reconstruction units configured to iteratively reconstruct an image.

4. The tomographic imaging system of claim 1, wherein each field programmable gate array comprises one or more pipelined processors.

5. The tomographic imaging system of claim 1, further comprising:
an X-ray source configured to emit X-rays;
a detector configured to generate electrical signals in response to the emitted X-rays; and
a data acquisition subsystem configured to acquire the electrical signals and to provide the electrical signals or a corresponding signal to the processing component.

6. The tomographic imaging system of claim 1, wherein the tomographic imaging system comprises at least one of a computed tomography imaging system, a positron emission tomography imaging system, a nuclear medicine imaging system, or a magnetic resonance imaging system.

7. A method for reconstructing a tomographic image, comprising:
processing an acquired image signal using one or more field programmable gate arrays configured as co-processors to a microprocessor to generate a reconstructed image signal.

8. The method of claim 7, wherein processing the acquired image signal comprises processing the acquired image signal with one or more reconstruction units running in parallel on each field programmable gate array.

9. The method of claim 7, wherein processing the acquired image signal comprises iteratively processing the acquired image signal with one or more reconstruction units on each field programmable gate array.

10. The method of claim 7, wherein processing the acquired image signal comprises processing the acquired image signal with one or more pipelined processors on each field programmable gate array.

11-19. (canceled)

20. A pipelined processor configured to forward- and back-project image data using the same data path and arithmetic units.

21. The pipelined processor of claim 20, wherein the pipelined processor is configured to process at least one pixeudetector boundary per clock cycle.

22. The pipelined processor of claim 20, wherein the pipelined processor is configured to implement one of a pixel-driven projection technique, a ray-driven projection technique, or a distance-driven projection technique.

23. A method for processing image data, comprising:
performing projection and back-projection operations on a pipelined processor, wherein the projection and back-projection operations employ the same data path and arithmetic units.

24. The method of claim 23, wherein the pipelined processor is configured to process at least one pixel/detector boundary per clock cycle.

25. The method of claim 23, wherein the projection and back-projection operations are based on one of a pixel-driven projection technique, a ray-driven projection technique, or a distance-driven projection technique.

26-34. (canceled)
